(19) United States
(12) Patent Application Publication
(10) Pub. No.: US 2007/0173908 A1
(43) Pub. Date: Jul. 26, 2007

(54) Transcutaneous trigeminal nerve stimulation to treat motion sickness

(75) Inventor: Jason D. Begnaud, Houston, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/336,684

(22) Filed: Jan. 20, 2006

(51) Int. Cl. A61N 1/18

(52) U.S. Cl. 607/63; 607/2

(57) ABSTRACT

A method of treating motion sickness in a patient is disclosed, wherein the method includes coupling at least one electrode to the patient at least one location in proximity to at least one cranial nerve; and applying an electrical signal to said nerve or nerve branch using said electrode to treat said motion sickness. An exemplary motion sickness is space motion sickness.

### BACKGROUND OF THE INVENTION

1. Field of the Invention

This invention relates generally to medical devices and, more particularly, to methods, apparatus, and systems for treating motion sickness, including space motion sickness, using stimulation of a cranial nerve.

2. Description of the Related Art

The human nervous system (HNS) includes the brain and the spinal cord, collectively known as the central nervous system (CNS). The central nervous system comprises nerve fibers that transmit nerves to, from, and within the brain and spinal cord. The network of nerves in the remaining portions of the human body forms the peripheral nervous system (PNS). Some peripheral nerves connect directly to the brain to control various brain functions, such as vision, eye movement, hearing, facial movement, and feeling. Another system of peripheral nerves, known as the autonomic nervous system (ANS), controls blood vessel diameter, intestinal movements, and actions of many internal organs. Autonomic functions include blood pressure, body temperature, heartbeat and essentially all the unconscious activities that occur without voluntary control.

Like the rest of the human nervous system, nerve signals travel up and down the peripheral nerves, which link the brain to the rest of the human body. Many, but not all, nerve fibers in the brain and the peripheral nerves are sheathed in a covering called myelin. The myelin sheath insulates electrical pulses traveling along the nerves. A nerve bundle may comprise up to 100,000 or more individual nerve fibers of different types, including larger diameter A and B fibers which comprise a myelin sheath and C fibers which have a much smaller diameter and are unmyelinated. Different types of nerve fibers, among other things, comprise different sizes, conduction velocities, stimulation thresholds, and myelination status (i.e., myelinated or unmyelinated).

The trigeminal nerve (cranial nerve V) innervates various regions of the face and mouth. A number of portions of various nerve branches of the trigeminal nerve, including the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve, pass in close proximity to the skin of the face and neck.

Motion sickness is a condition produced by confusion of the vestibular (balance) system resulting from travel in a vehicle, such as an automobile, a boat, an airplane, or a spacecraft. Common symptoms of motion sickness include nausea and vomiting. Motion sickness in spacecraft may also be referred to as microgravity nausea or space motion sickness (SMS). High percentages of astronauts have either experienced SMS at some point in their mission or continue to experience the symptoms throughout several days of the mission while in spaceflight. SMS tends to hinder their productivity during the highly scheduled missions required of spacecraft crews.

The present invention is directed to overcoming, or at least reducing, the effects of one or more of the problems set forth above.

### SUMMARY OF THE INVENTION

In one aspect, the present invention comprises a method for treating motion sickness in a patient, including coupling at least one electrode to a cranial nerve of a patient, and applying an electrical signal to the nerve using said at least one electrode to treat the motion sickness. In particular embodiments, the cranial nerve may be a trigeminal nerve, a vagus nerve, or a glossopharyngeal nerve. In a specific embodiment, the at least one electrode may be coupled to the trigeminal nerve or a branch of the trigeminal nerve selected from the group consisting of the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve. The electrode may be coupled to the patient's skin to provide transcutaneous, indirect stimulation to the trigeminal nerve in one embodiment. In another embodiment, the electrode may be placed subcutaneously near or on a branch of the trigeminal nerve to provide indirect or direct stimulation. In yet another embodiment, the electrode may be coupled to directly to a main branch or ganglion of the trigeminal nerve in the patient's cranium.

In another aspect, the present invention comprises a method for treating motion sickness in a patient, including coupling at least one electrode to the skin of the patient at at least one location in proximity to at least one nerve branch selected from the group consisting of the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve; and applying an electrical signal to said nerve branch using said electrode to treat said motion sickness.

In one aspect, the motion sickness is induced by automobile travel, boat travel, aircraft travel, or spacecraft travel.

In a further aspect, the present invention comprises a method for treating motion sickness in a patient, including coupling at least one electrode to an autonomic nerve of the patient selected from the group consisting of the trigeminal nerve, the vagus nerve, the glossopharyngeal nerve, the sympathetic nerve, and branches of the foregoing, and applying an electrical signal to the nerve using said at least one electrode to treat the motion sickness.

### BRIEF DESCRIPTION OF THE DRAWINGS

The invention may be understood by reference to the following description taken in conjunction with the accompanying drawings, in which like reference numerals identify like elements, and in which:

FIG. 1 is a stylized schematic representation of a medical device that stimulates a cranial nerve for treating a patient with motion sickness, according to one illustrative embodiment of the present invention;

FIG. 2 is a stylized schematic representation of a medical device that stimulates the various branches of the trigeminal nerve from the skin electrode locations for treating a patient with motion sickness, according to one illustrative embodiment of the present invention;

FIG. 3A illustrates an exemplary electrical signal of a firing neuron as a graph of voltage at a given location at particular times during firing by the neurostimulator of FIG. 1, when applying an electrical signal to the autonomic nerves, in accordance with one illustrative embodiment of the present invention;

FIG. 3B illustrates an exemplary electrical signal response of a firing neuron as a graph of voltage at a given location at particular times during firing by the neurostimulator of FIG. 1, when applying a sub-threshold depolarizing pulse and additional stimulus to the trigeminal nerve, in accordance with one illustrative embodiment of the present invention;

FIG. 3C illustrates an exemplary stimulus including a sub-threshold depolarizing pulse and additional stimulus to the trigeminal nerve for firing a neuron as a graph of voltage at a given location at particular times by the neurostimulator of FIG. 1, in accordance with one illustrative embodiment of the present invention;

FIGS. 4A, 4B, and 4C illustrate exemplary waveforms for generating the electrical signals for stimulating the trigeminal nerve for treating motion sickness, according to one illustrative embodiment of the present invention;

FIG. 5 illustrates a stylized block diagram depiction of the medical device for treating motion sickness, in accordance with one illustrative embodiment of the present invention;

FIG. 6 illustrates a flowchart depiction of a method for treating motion sickness, in accordance with illustrative embodiment of the present invention;

FIG. 7 illustrates a flowchart depiction of an alternative method for treating motion sickness, in accordance with an alternative illustrative embodiment of the present invention;

FIG. 8 depicts a more detailed flowchart depiction of step of performing a detection process of FIG. 7, in accordance with an illustrative embodiment of the present invention; and

FIG. 9 depicts a more detailed flowchart depiction of the steps of determining a particular type of stimulation based upon data relating to motion sickness described in FIG. 7, in accordance with an illustrative embodiment of the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF PARTICULAR EMBODIMENTS

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

Certain terms are used throughout the following description and claims to refer to particular system components. As one skilled in the art will appreciate, components may be referred to by different names. This document does not intend to distinguish between components that differ in name but not function. In the following discussion and in the claims, the terms “include” and “including” are used in an open-ended fashion, and thus should be interpreted to mean “including, but not limited to.” Also, the term “couple” or “couples” is intended to mean either a direct or an indirect electrical connection. For example, if a first device couples to a second device, that connection may be through a direct electrical connection or through an indirect electrical connection via other devices, biological tissues, or magnetic fields. “Direct contact,” “direct attachment,” or providing a “direct coupling” indicates that a surface of a first element contacts the surface of a second element with no substantial attenuating medium therebetween. The presence of substances, such as bodily fluids, that do not substantially attenuate electrical connections does not vitiate direct contact. “Transcutaneous contact” or variations thereof indicates that a surface of a first element contacts the skin of a patient and does not directly contact the surface of a second element on the other side of the skin of the patient. The word “or” is used in the inclusive sense (i.e., “and/or”) unless a specific use to the contrary is explicitly stated.

Embodiments of the present invention provide for the treatment of motion sickness by stimulation of autonomic nerves, such as the trigeminal nerve, the vagus nerve, other cranial nerves, the sympathetic nerve, or branches of the foregoing. Embodiments of the present invention provide for an electrical stimulation for a portion of an autonomic nerve to treat motion sickness. Motion sickness may be treated utilizing the electrical stimulation provided by a medical device. As used herein, stimulation refers to the application of an electrical signal to the nerve. The electrical signal may induce afferent and/or efferent action potentials on the nerve, may block native afferent and/or efferent action potentials, or may be applied at a sub-threshold level that neither generates action potentials nor blocks native action potentials. In preferred embodiments, the electrical signal is a signal that is capable of inducing afferent and/or efferent action potentials on the nerve.

In one embodiment, application of the stimulation signal may be designed to promote a blocking effect relating to a signal that is being sent from the brain to the various portions of the gastrointestinal system to treat motion sickness. This may be accomplished by delivering a particular type of controlled electrical signal, such as a controlled current signal to the autonomic nerve.

Cranial nerve stimulation has been used successfully to treat a number of nervous system disorders, including epilepsy and other movement disorders, depression and other neuropsychiatric disorders, dementia, coma, migraine headache, obesity, eating disorders, sleep disorders, cardiac disorders (such as congestive heart failure and atrial fibrillation), hypertension, endocrine disorders (such as diabetes and hypoglycemia), and pain, among others. See, e.g., U.S. Pats. Nos. 4,867,164; 5,299,569; 5,269,303; 5,571,150; 5,215,086; 5,188,104; 5,263,480; 6,587,719; 6,609,025; 5,335,657; 6,622,041; 5,916,239; 5,707,400; 5,231,988; and 5,330,515. Despite the recognition that cranial nerve stimulation may be an appropriate treatment for the foregoing conditions, the fact that detailed neural pathways for many (if not all) cranial nerves remain relatively unknown makes predictions of efficacy for any given disorder difficult. Even if such pathways were known, moreover, the precise stimulation parameters that would energize particular pathways that affect the particular disorder likewise are difficult to predict. Accordingly, cranial nerve stimulation, and particularly transcutaneous trigeminal nerve stimulation, has not heretofore been deemed appropriate for use in treating motion sickness.

In one embodiment of the present invention, methods, apparatus, and systems transcutaneously stimulate an autonomic nerve, such as a cranial nerve, e.g., a trigeminal nerve, using an electrical signal to treat motion sickness. The “electrical signal” applied to the nerve in embodiments of the present invention refers to an exogenous electrical signal that is distinct from the endogenous electrical activity (i.e., afferent and/or efferent action potentials) generated by the patient's body and environment. In other words, the electrical signal applied to the nerve in the present invention is a signal applied from an artificial source, e.g., a neurostimulator. In a particular embodiment of the present invention, a method for treating motion sickness is provided using stimulation of the trigeminal nerve (cranial nerve V), and preferably the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve. Certain parameters defining the electrical signal generated by the neurostimulator are programmable, such as by means of an external programmer in a manner conventional for electrical medical devices, or by manual adjustment, depending upon the design of the specific neurostimulator used.

More generally, embodiments of the present invention provide for an electrical stimulation of a portion of an autonomic nerve to treat motion sickness. An electrical signal may be applied to a portion of a cranial nerve (e.g., a trigeminal nerve, vagus nerve, or glossopharyngeal nerve), such as the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve, to affect motion sickness. Additionally, the electrical signal may be such as to induce afferent, efferent and/or afferent-efferent combination action potentials to treat motion sickness. The electrical signal may also be such as to block electrical activity on the nerve. In still other embodiments, the electrical signal may comprise a plurality of signals, some of which block electrical activity on the nerve and some of which generate afferent and/or efferent action potentials on the nerve

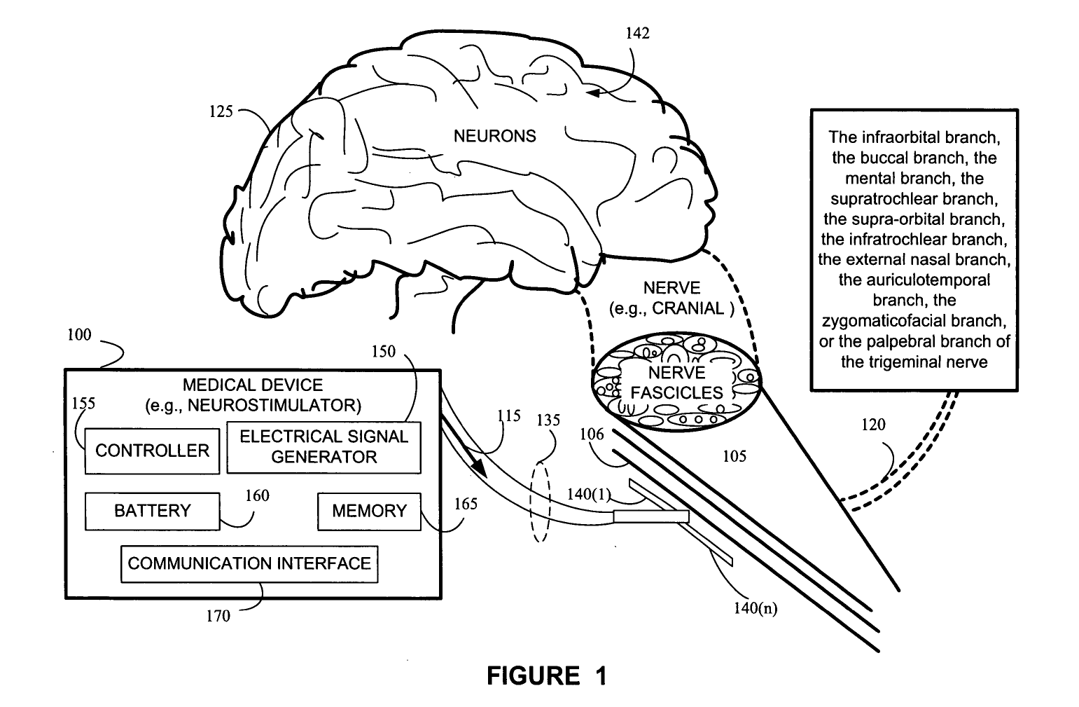

Turning now to FIG. 1, a medical device 100 is provided for electrically stimulating a nerve, such as an autonomic nerve 105 of a patient to treat motion sickness, according to one illustrative embodiment of the present invention. The medical device 100 may be an implantable medical device (IMD) or may comprise a device that is located external to the body of the patient. Medical device 100 may be a self-contained device with controls integral to the device or may include a separate control unit for programming and controlling the delivery of the electrical signal 115 to the autonomic nerve 100. The term “autonomic nerve” refers to any portion of the main trunk or any branch of a peripheral nerve that regulates or affects autonomic functions of the body. Specific autonomic nerves include the trigeminal nerve, the vagus nerve, other cranial nerves, the sympathetic nerve, or branches of the foregoing. The medical device 100 may deliver an electrical signal 115 to a nerve branch 120 of the autonomic nerve 105 that travels to the brain 125 of a patient. The nerve branch 120 may be associated with the parasympathetic control and/or the sympathetic control of the vomiting/nausea function for motion sickness.

The medical device 100 may apply neurostimulation by delivering the electrical signal 115 to the nerve branch 120 via a lead 135 coupled to one or more electrodes 140 (1-n) sited in proximity to the patient's skin 106. For example, the medical device 100 may stimulate the autonomic nerve 105 by applying the electrical signal 115 to the nerve branch 120 that transcutaneously contacts the main trunk of the right and/or left trigeminal nerve, the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve, using the electrode(s) 140(1-n).

Consistent with one embodiment of the present invention, the medical device 100 may be a neurostimulator device capable of treating a disease, disorder or condition relating to the vomiting/nausea functions of a patient by providing electrical neurostimulation therapy to a patient. In order to accomplish this task, the medical device 100 may be located on the skin of the patient at a suitable site in proximity to a portion of a branch of the trigeminal nerve or other cranial nerve, such as the vagus nerve. The medical device 100 may apply the electrical signal 115, which may comprise a pulsed electrical signal, to the autonomic nerve 105. The medical device 100 may generate the electrical signal 115 defined by one or more parameters. These parameters may be programmed to one or more desired values within a predetermined range. The medical device 100 may apply the electrical signal 115 to the nerve branch 120 or a nerve fascicle within the autonomic nerve 105. By applying the electrical signal 115, the medical device 100 may treat motion sickness in a patient.

Medical devices 100 that may be used in the present invention include any of a variety of electrical stimulation devices, such as a neurostimulator capable of stimulating a neural structure in a patient, especially for stimulating a patient's autonomic nerve, such as a trigeminal nerve. The medical device 100 preferably is capable of delivering a controlled current stimulation signal. Although the medical device 100 is described in terms of autonomic nerve stimulation, and particularly trigeminal nerve stimulation (TNS), a person of ordinary skill in the art would recognize that the present invention is not so limited. For example, the medical device 100 may be applied to the stimulation of other autonomic nerves, including sympathetic or parasympathetic nerves, and/or other neural tissue in the peripheral nervous system. While not preferred, in some embodiments, the stimulation may be delivered to portions of the CNS, and specifically to portions of the patient's brain.

Applying the electrical signal 115 to a selected autonomic nerve 105 may comprise generating a response in the nerve selected from the group consisting of an afferent action potential, an efferent action potential, an afferent hyperpolarization, an efferent hyperpolarization, and blocking afferent and efferent action potentials. In a preferred embodiment, the medical device 100 may generate an afferent action potential for treating motion sickness.

The medical device 100 may comprise an electrical signal generator 150 and a controller 155 operatively coupled thereto to generate the electrical signal 115 for causing the nerve stimulation. The stimulus generator 150 may generate the electrical signal 115. The controller 155 may be adapted to apply the electrical signal 115 to the autonomic nerve 105 to provide electrical neurostimulation therapy to the patient for treating motion sickness. The controller 155 may direct the stimulus generator 150 to generate the electrical signal 115 to stimulate the autonomic nerve 105.

To generate the electrical signal 115, the medical device 100 may further include a power supply, such as a battery 160, a memory 165, and a communication interface 170. More specifically, the battery 160 may comprise a power-source battery that may be rechargeable. The battery 160 provides power for the operation of the medical device 100, including electronic operations and the stimulation function. The battery 160, in one embodiment, may be a lithium/thionyl chloride cell or, in another embodiment, a lithium/carbon monofluoride cell. In another embodiment, the battery 160 may be an alkaline cell or a nickel/cadmium cell. In another embodiment, the power supply may be a generator such as those typically present in automobiles, boats, ships, aircraft, or spacecraft. The memory 165, in one embodiment, is capable of storing various data, such as operation parameter data, status data, and the like, as well as program code. The communication interface 170 is capable of providing transmission and reception of electronic signals to and from an external unit. Adjustment of the controller 155 can also be performed manually by external switches or knobs directly coupled to the controller or by external interface 170. The external unit may be a device that is capable of programming the medical device 100.

In one embodiment, the medical device 100, which may be a single device or a plurality of devices, is electrically coupled to the lead(s) 135, which are in turn coupled to the electrode(s) 140 transcutaneously contacted to the left and/or right branches of the trigeminal nerve, such as the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve, for example. In certain embodiments, the electrodes may be integrally formed as part of medical device 100, and lead(s) 135 may be omitted. If multiple electrode(s) 140 are used, two or more of the multiple electrodes may be coupled to a single nerve branch or each electrode may be coupled to a different nerve branch.

The medical device 100 may also include sensing electrodes (i.e., electrodes that sense one or more body parameters) as well as stimulating electrodes to deliver electrical signal 115. In one embodiment, the electrode(s) 140 (1-n) may include a set of stimulating electrode(s) separate from a set of sensing electrode(s). In another embodiment, the same electrode(s) may be deployed to stimulate and to sense. A particular type or a combination of electrodes may be selected as desired for a given application. For example, an electrode suitable for transcutaneous coupling to a trigeminal nerve may be used. The electrodes 140 may comprise a bipolar stimulating electrode pair. Those skilled in the art having the benefit of the present invention will appreciate that many electrode designs could be used in the present invention.

Using the electrode(s) 140(1-n), the stimulus generator 150 may apply a predetermined electrical signal 115 to the selected autonomic nerve 105 to provide therapeutic neurostimulation for the patient with motion sickness. While the selected autonomic nerve 105 may be the trigeminal nerve, the electrode(s) 140(1-n) may comprise at least one electrode capable of transcutaneous contact with the patient's trigeminal nerve for indirect stimulation thereof. Alternatively, an electrode may be placed subcutaneously and in proximity to the patient's trigeminal nerve or a branch thereof for direct stimulation of the nerve or branch.

A particular embodiment of the medical device 100 may be a programmable electrical signal generator. Such a programmable electrical signal generator may be capable of programmatically defining the electrical signal 115. By providing an electrical signal defined by at least one parameter selected from the group consisting of a current magnitude, a pulse frequency, and a pulse width, the medical device 100 may treat motion sickness. The medical device 100 may detect a symptom of motion sickness. In response to detecting the symptom, the medical device 100 may initiate applying the electrical signal 115. For example, a sensor may be used to detect the symptom of motion sickness. To treat motion sickness, the medical device 100 may apply the electrical signal 115 by applying a first electrical signal during a first treatment period and further applying a second electrical signal to the autonomic nerve 105 using the electrode 140 during a second treatment period.

In one embodiment, the method may further include detecting a symptom of motion sickness, wherein applying the electrical signal 115 to the autonomic nerve 105 is initiated in response to the detecting of the symptom. In a further embodiment, detecting the symptom may be performed by the patient. This may involve a subjective observation that the patient is experiencing a symptom of motion sickness, such as nausea, followed by an external input from the patient to the medical device 100.

The method may be performed under a single treatment regimen or under multiple treatment regimens. “Treatment regimen” herein may refer to a parameter of the electrical signal 115, a duration for applying the signal, and/or a duty cycle of the signal, among others. In one embodiment, applying the electrical signal 115 to the autonomic nerve 105 is performed by applying a first electrical signal during a first treatment period, and may further include the step of applying a second electrical signal, different from the first electrical signal, to the cranial nerve using the electrode 140 during a second treatment period. In a further embodiment, the method may include detecting a symptom of motion sickness, wherein the second treatment period is initiated upon the detection of the symptom. The patient may benefit by receiving a first electrical signal during a first, acute treatment period and a second electrical signal during a second, chronic treatment period. Three or more treatment periods may be used, if deemed desirable by a medical practitioner. In an alternative embodiment, a plurality of different electrical signals, each having at least one parameter defining the signal that is different from the other signals, may be applied to the nerve during a single treatment period. This may include alternating between two, three, or more electrical signals.

If multiple electrodes 140 are used, the first treatment period may involve applying the first electrical signal to a nerve branch using a first electrode and the second treatment period may involve applying the second treatment period to the same or a different nerve branch using a second electrode.

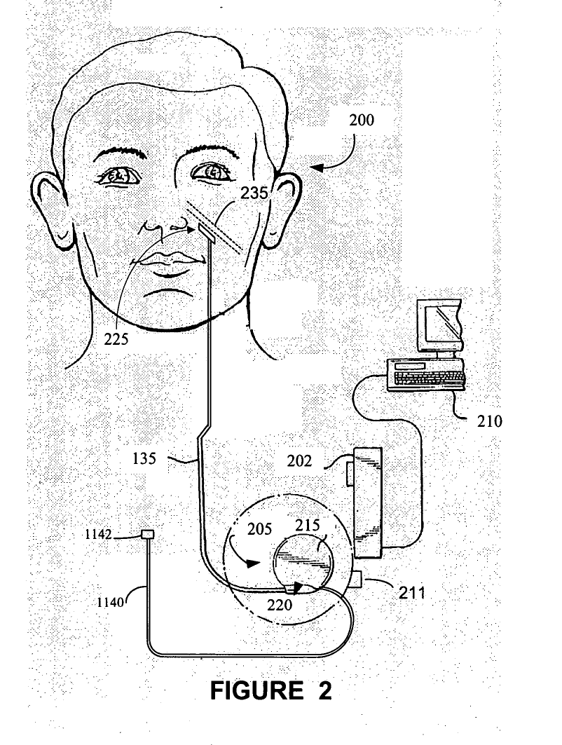

As shown in FIG. 2, an external programming user interface 202 may be used by a health professional for a particular patient to either initially program and/or to later reprogram the medical device 100, such as a neurostimulator 205. The neurostimulator 205 may include the electrical signal generator 150, which may be programmable. To enable physician programming of the electrical and timing parameters of a sequence of electrical impulses, a programming system 210 may be indirectly coupled, e.g., via RF communication, to neurostimulator 205. The external programming system 210 may include a processor-based computing device, such as a computer, personal digital assistant (PDA) device, or other suitable computing device. In embodiments wherein the neurostimulator 205 is located externally to the patient's body during use, programming of the neurostimulator 205 may alternatively, or in addition to external system 210, be performed directly by switches, knobs, buttons, keyboard inputs, or other known means 211 that are integral to the neurostimulator 205.

Using the external programming user interface 202 or external switches, knobs, or buttons 211, a user of the external programming system 210 may program the neurostimulator 205. Communications between the neurostimulator 205 and the external programming system 210 may be accomplished using any of a variety of conventional techniques known in the art. The neurostimulator 205 may include a transceiver (such as a coil) that permits signals to be communicated wirelessly between the external programming user interface 202, such as a wand, and the neurostimulator 205.

The neurostimulator 205 may comprise a case 215 with an electrically conducting connector on header 220, and in some embodiments may be worn in a pocket of the patient's clothing or be secured to the body by, e.g., a combination of straps and hook-and-loop fabric similar to an armband for MP3 players as is known in the art. A stimulating nerve electrode assembly 225, preferably comprising an electrode pair, is conductively connected to the distal end of an insulated electrically conductive lead assembly 135, which preferably comprises a pair of lead wires and is attached at its proximal end to the connector on the case 215. The electrode assembly 225 is transcutaneously coupled to a portion of the trigeminal nerve 235 on the skin of the patient's face. Persons of skill in the art will appreciate that many electrode designs could be used in the present invention.

In one embodiment, the electrode(s) 140 (1-n) of medical device 100 (FIG. 1) may sense or detect any target symptom parameter in the patient's body 200. For example, an electrode 140 transcutaneously coupled to the patient's trigeminal nerve may detect a factor associated with motion sickness. For example, a sensor or any other element capable of providing a sensing signal representative of a patient's body parameter associated with motion sickness may be deployed, such as sensor 1142, which may communicate with the neurostimulator 205 via lead 1140 (FIG. 2).

In one embodiment, the neurostimulator 205 may be programmed to deliver an electrical biasing signal at programmed time intervals (e.g., every five minutes). In an alternative embodiment, the neurostimulator 205 may be programmed to initiate an electrical biasing signal upon detection of an event or upon another occurrence to deliver therapy. Based on this detection, a programmed therapy may be determined to the patient in response to signal(s) received from one or more sensors indicative of corresponding monitored patient parameters.

The electrode(s) 140(1-n), as shown in FIG. 1 may be used in some embodiments of the invention to trigger administration of the electrical stimulation therapy to the trigeminal nerve 235 via electrode assembly 225. Use of such sensed body signals to trigger or initiate stimulation therapy is hereinafter referred to as “active,” “triggered,” or “feedback” modes of administration. Other embodiments of the present invention utilize a continuous, periodic or intermittent stimulus signal. These signals may be applied to the trigeminal nerve (each of which constitutes a form of continual application of the signal) according to a programmed on/off duty cycle. In one embodiment, zero sensors may be used to trigger therapy delivery. This type of delivery may be referred to as a “passive” or “prophylactic” therapy mode. Both active and passive electrical biasing signals may be combined or delivered by a single neurostimulator according to the present invention.

The electrical signal generator 150 may be programmed using programming software of the type copyrighted by the assignee of the instant application with the Register of Copyrights, Library of Congress, or other suitable software based on the description herein. A programming wand (not shown) may be used to facilitate radio frequency (RF) communication between the external progranming user interface 202 and the electrical signal generator 150. The wand and software permit noninvasive communication with the electrical signal generator 150 after the neurostimulator 205 is worn. The wand may be powered by internal batteries, and provided with a “power on” light to indicate sufficient power for communication. Another indicator light may be provided to show that data transmission is occurring between the wand and the neurostimulator 205. Alternatively, the electrical signal generator 150 can contain a programming user interface.

The neurostimulator 205 may provide trigeminal nerve stimulation (TNS) therapy upon a trigeminal nerve branch and/or to any portion of the autonomic nervous system. The neurostimulator 205 may be activated manually or automatically to deliver the electrical bias signal to the selected cranial nerve via the electrode 140. The neurostimulator 205 may be programmed to deliver the electrical signal 115 continuously, periodically or intermittently when activated.

In one embodiment, stimulation may be applied so as to generate afferent action potentials, which refers to signals traveling on a nerve in a direction toward the central nervous system. In another embodiment, a “blocking” type of stimulation may be employed using the medical device 100, such that efferent fibers in communication with a locus in the central nervous system which is associated with a symptom of motion sickness, such as the nucleus tractus solitarius (NTS), and to which the afferent fibers of the cranial nerve project are not stimulated.

The electrical stimulation treatment described herein may be used to treat motion sickness separately, or in combination with another type of treatment. For example, electrical stimulation treatment may be applied in combination with a chemical agent, such as various drugs, to treat motion sickness. The electrical stimulation treatment may also be performed in combination with other types of treatment, such as magnetic stimulation treatment and/or biological treatments. Combining the electrical stimulation with the chemical, magnetic, and/or biological treatments, side effects associated with certain drugs and/or biological agents may be reduced.

In one embodiment, chemical treatment may include further coupling a drug delivery device in direct or indirect fluid communication with the patient's bloodstream and applying a drug to said bloodstream using said drug delivery device to treat said motion sickness. The drug delivery device may be a drug pump or a transcutaneous patch, among others. In one embodiment, the drug may be selected from the group consisting of promethazine, dimenhydrinate, diphenhydramine, meclizine, cyclizine, diazepam, and scopolamine.

In addition or alternatively to afferent fiber depolarization, afferent fiber hyperpolarization may be performed to treat motion sickness.

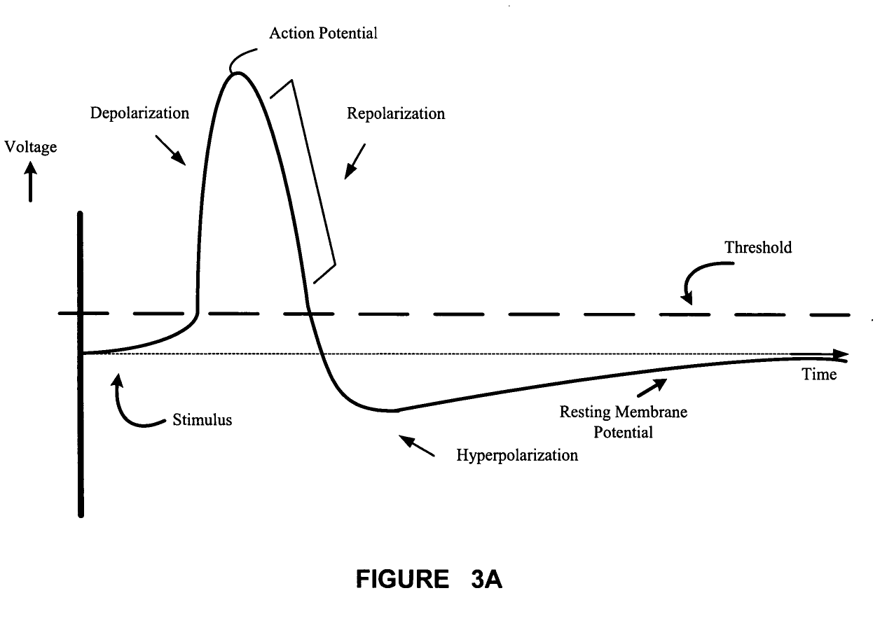

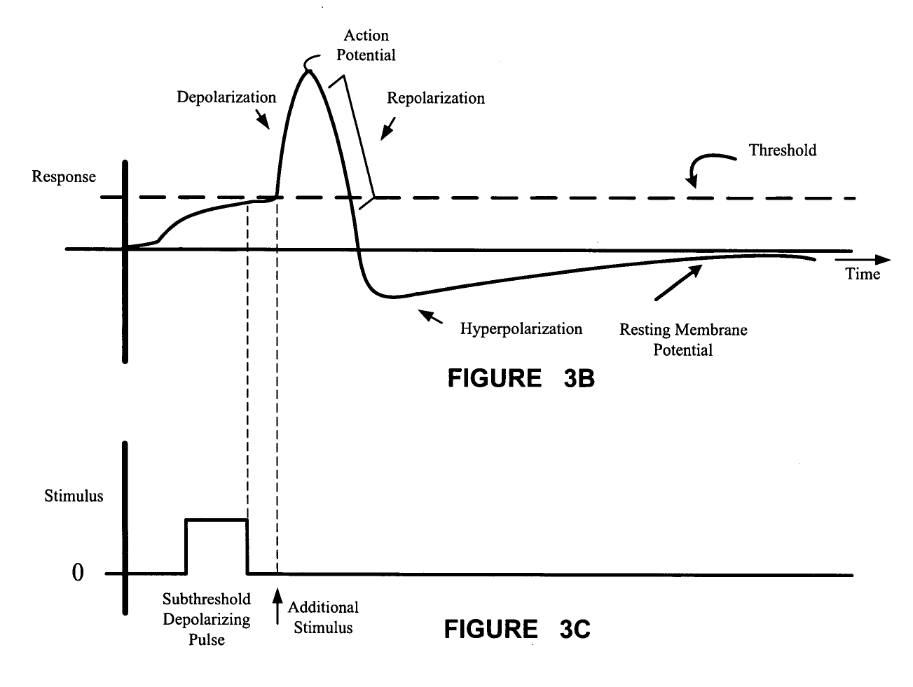

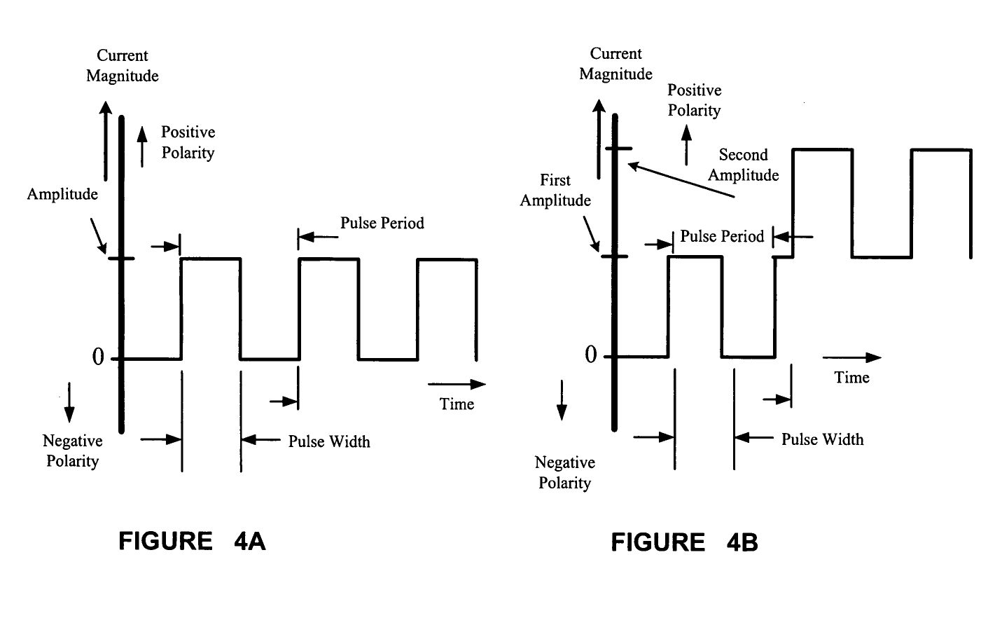

FIG. 3A provides a stylized depiction of an exemplary electrical signal of a firing neuron as a graph of voltage at a given location at particular times during firing, in accordance with one embodiment of the present invention. A typical neuron has a resting membrane potential of about −70 mV, maintained by transmembrane ion channel proteins. When a portion of the neuron reaches a firing threshold of about −55 mV, the ion channel proteins in the locality allow the rapid ingress of extracellular sodium ions, which depolarizes the membrane to about +30 mV. The wave of depolarization then propagates along the neuron. After depolarization at a given location, potassium ion channels open to allow intracellular potassium ions to exit the cell, lowering the membrane potential to about −80 mV (hyperpolarization). About 1 msec is required for transmembrane proteins to return sodium and potassium ions to their starting intra- and extracellular concentrations and allow a subsequent action potential to occur. The present invention may raise or lower the resting membrane potential, thus making the reaching of the firing threshold more or less likely and subsequently increasing or decreasing the rate of fire of any particular neuron.

Referring to FIG. 3B, an exemplary electrical signal response is illustrated of a firing neuron as a graph of voltage at a given location at particular times during firing by the neurostimulator of FIG. 2, in accordance with one illustrative embodiment of the present invention. As shown in FIG. 3C, an exemplary stimulus including a sub-threshold depolarizing pulse and additional stimulus to the autonomic nerve 105, such as the trigeminal nerve 235 may be applied for firing a neuron, in accordance with one illustrative embodiment of the present invention. The stimulus illustrated in FIG. 3C depicts a graph of voltage at a given location at particular times by the neurostimulator of FIG. 2.

The neurostimulator may apply the stimulus voltage of FIG. 3C to the autonomic nerve 105, which may include afferent fibers, efferent fibers, or both. This stimulus voltage may cause the response voltage shown in FIG. 3B. Afferent fibers transmit information to the brain from the extremities; efferent fibers transmit information to the extremities from the brain. The trigeminal nerve 235 may include both afferent and efferent fibers, and the neurostimulator 205 may be used to stimulate either or both. In one embodiment, the neurostimulator 205 may be used to stimulate afferent fibers of the trigeminal nerve 235.

The autonomic nerve 105 may include fibers that transmit information in the sympathetic nervous system, the parasympathetic nervous system, or both. Inducing an action potential in the sympathetic nervous system may yield a result similar to that produced by blocking an action potential in the parasympathetic nervous system and vice versa.

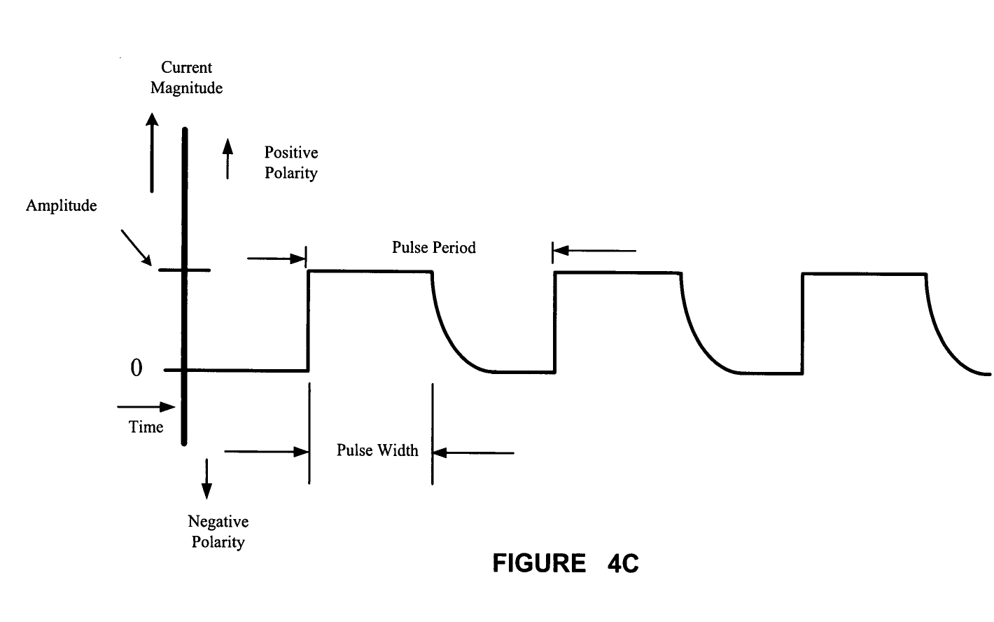

Referring back to FIG. 2, the neurostimulator 205 may generate the electrical signal 115 according to one or more programmed parameters for stimulation of the trigeminal nerve 235. In one embodiment, the stimulation parameter may be selected from the group consisting of a current magnitude, a pulse frequency, a signal width, on-time, and off-time. An exemplary table of ranges for each of these stimulation parameters is provided in Table 1. The stimulation parameter may be of any suitable waveform; exemplary waveforms in accordance with one embodiment of the present invention are shown in FIGS. 4A-4C. Specifically, the exemplary waveforms illustrated in FIGS. 4A-4C depict the generation of the electrical signal 115 that may be defined by a factor related to at least one condition relating to motion sickness, relative to a value within a defined range.

According to one illustrative embodiment of the present invention, various electrical signal patterns may be employed by the neurostimulator 205. These electrical signals may include a plurality of types of pulses, e.g., pulses with varying amplitudes, polarity, frequency, etc. For example, the exemplary waveform 4A depicts that the electrical signal 115 may be defined by fixed amplitude, constant polarity, pulse width, and pulse period. The exemplary waveform 4B depicts that the electrical signal 115 may be defined by a variable amplitude, constant polarity, pulse width, and pulse period. The exemplary waveform 4C depicts that the electrical signal 115 may be defined by a fixed amplitude pulse with a relatively slowly discharging current magnitude, constant polarity, pulse width, and pulse period. Other types of signals may also be used, such as sinusoidal waveforms, etc. The electrical signal may be controlled current signals.

TABLE 1

| PARAMETER | RANGE | |
|---|---|---|
| Output current | 0.1-12.0 | mA |
| Pulse width | 10-1500 | μsec |
| Frequency | 0.5-250 | Hz |
| On-time | 1 | sec and greater |
| Off-time | 0 | sec and greater |
| Frequency Sweep | 1-100 | Hz |
| Random Frequency | 1-100 | Hz |

On-time and off-time parameters may be used to define an intermittent pattern in which a repeating series of signals may be generated for stimulating the nerve 105 during the on-time. Such a sequence may be referred to as a “pulse burst.” This sequence may be followed by a period in which no signals are generated. During this period, the nerve is allowed to recover from the stimulation during the pulse burst. The on/off duty cycle of these alternating periods of stimulation and idle periods may have a ratio in which the off-time may be set to zero, providing continuous stimulation. Alternatively, the idle time may be as long as one day or more, in which case the stimulation is provided once per day or at even longer intervals. Typically, however, the ratio of “off-time” to “on-time” may range from about 0.5 to about 10.

In one embodiment, the width of each signal may be set to a value not greater than about 1.5 msec, such as about 250-500 μsec, and the signal repetition frequency may be programmed to be in a range of about 20-250 Hz. In one embodiment, a frequency of 150 Hz may be used. A non-uniform frequency may also be used. Frequency may be altered during a pulse burst by either a frequency sweep from a low frequency to a high frequency, or vice versa. Alternatively, the timing between adjacent individual signals within a burst may be randomly changed such that two adjacent signals may be generated at any frequency within a range of frequencies.

In one embodiment, the present invention may include coupling of at least one electrode to each of two or more cranial nerves or branches thereof. (In this context, two or more cranial nerves mean two or more nerves having different names or numerical designations, and do not refer to the left and right versions of a particular nerve). In one embodiment, at least one electrode 140 may be coupled to each of the trigeminal nerve 235 and/or a branch of the trigeminal nerve. Each of the nerves in this embodiment or others involving two or more cranial nerves may be stimulated according to particular activation modalities that may be independent between the two nerves.

Another activation modality for stimulation is to program the output of the neurostimulator 205 to the maximum amplitude that the patient may tolerate. The stimulation may be cycled on and off for a predetermined period of time followed by a relatively long interval without stimulation. Where the cranial nerve stimulation system is completely external to the patient's body, higher current amplitudes may be needed to overcome the attenuation resulting from the absence of direct contact with the trigeminal nerve 235 and the additional impedance of the skin of the patient. Although external systems typically require greater power consumption than implantable ones, they have an advantage in that their batteries may be more easily replaced. Also, in treating motion sickness, the duration of the treatment regimen(s) can be relatively short, from a few hours for automobile or airplane travel to up to about a week or longer for an oceangoing voyage or a manned spaceflight mission.

Other types of indirect stimulations may be performed in conjunction with embodiments of the invention. In one embodiment, the invention includes providing noninvasive transcranial magnetic stimulation (TMS) to the brain 125 of the patient along with the medical device 100 of the present information to treat motion sickness. TMS systems include those disclosed in U.S. Pats. Nos. 5,769,778; 6,132,361; and 6,425,852. Where TMS is used, it may be used in conjunction with cranial nerve stimulation as an adjunctive therapy. In one embodiment, both TMS and cranial nerve stimulation may be performed to treat motion sickness. Other types of stimulation, such as chemical stimulation to treat motion sickness may be performed in combination with the medical device 100.

Returning to systems for providing autonomic nerve stimulation, such as that shown in FIGS. 1 and 2, stimulation may be provided in at least two different modalities. Where cranial nerve stimulation is provided based solely on programmed off-times and on-times, the stimulation may be referred to as passive, inactive, or non-feedback stimulation. In contrast, stimulation may be triggered by one or more feedback loops according to changes in the body or mind of the patient. This stimulation may be referred to as active or feedback-loop stimulation. In one embodiment, feedback-loop stimulation may be manually-triggered stimulation, in which the patient manually causes the activation of a pulse burst outside of the programmed on-time/off-time cycle. The patient may manually activate the neurostimulator 205 to stimulate the autonomic nerve 105 to treat the acute episode of motion sickness. The patient may also be permitted to alter the intensity of the signals applied to the autonomic nerve within limits established by the physician. For example, the patient may be permitted to alter the signal frequency, current, duty cycle, or a combination thereof. In at least some embodiments, the neurostimulator 205 may be programmed to generate the stimulus for a relatively long period of time in response to manual activation.

Patient activation of a neurostimulator 205 may involve use of an external control magnet, a sensor such as a piezoelectric element mounted to the inner surface of the generator case and adapted to detect light taps by the patient, or buttons or switches on the neurostimulator and accessible by hand to the patient. One or more taps applied in a predetermined sequence to the electrical signal generator 150 may be programmed into the medical device 100 as a signal for activation of the electrical signal generator 150. Two taps spaced apart by a slightly longer duration of time may be programmed into the medical device 100 to indicate a desire to deactivate the electrical signal generator 150, for example. The patient may be given limited control over operation of the device to an extent that may be determined by the program dictated or entered by the attending physician. The patient may also activate the neurostimulator 205 using other suitable techniques or apparatus.

In some embodiments, feedback stimulation systems other than manually-initiated stimulation may be used in the present invention. An autonomic nerve stimulation system may include a sensing lead coupled at its proximal end to a header along with a stimulation lead and electrode assemblies. A sensor may be coupled to the distal end of the sensing lead. The sensor may include a temperature sensor, a motion sickness parameter sensor (e.g., a sensor to detect a physiological factor indicative of motion sickness), a heart parameter sensor, a brain parameter sensor, or a sensor for another body parameter. The sensor may also include a nerve sensor for sensing activity on a nerve, such as a cranial nerve, such as the trigeminal nerve 235.

In one embodiment, the sensor may sense a body parameter that corresponds to a symptom of motion sickness. If the sensor is to be used to detect a symptom of the medical disorder, a signal analysis circuit may be incorporated into the neurostimulator 205 for processing and analyzing signals from the sensor. Upon detection of the symptom of motion sickness, the processed digital signal may be supplied to a microprocessor in the neurostimulator 205 to trigger application of the electrical signal 115 to the autonomic nerve 105. In another embodiment, the detection of a symptom of interest may trigger a stimulation program including a stimulation process that employs different stimulation parameters from a passive stimulation program. This may entail providing a higher current stimulation signal or providing a higher ratio of on-time to off-time.

In response to the afferent action potentials, a detection unit 695 may detect an indication of change in the symptom characteristic. The detection unit 695 may provide feedback to the medical device 100 to provide an indication of change in the symptom characteristic, which may be used to modulate the electrical signal 115. In response to providing feedback for the indication, the electrical signal generator 150 may adjust the afferent action potentials to enhance efficacy of a drug in the patient.

The neurostimulator 205 may use the memory 165 to store disorder data and a routine to analyze this data. The disorder data may include sensed body parameters or signals indicative of the sensed parameters. The routine may comprise software and/or firmware instructions to analyze the sensed hormonal activity for determining whether electrical neurostimulation would be desirable. If the routine determines that electrical neurostimulation is desired, then the neurostimulator 205 may provide an appropriate electrical signal to a neural structure, such as the trigeminal nerve 235.

In certain embodiments, the medical device 100 may comprise a neurostimulator 205 having a case 215 as a main body in which the electronics described in FIGS. 1-2 may be enclosed and sealed. Coupled to the main body may be the header 220 designed with terminal connectors for connecting to a proximal end of the electrically conductive lead(s) 135. The lead(s) 135 projecting from the electrically conductive lead assembly 230 of the header may be coupled at a distal end to electrodes 140(1-n). The electrodes 140(1-n) may be transcutaneously coupled to neural structure such as the trigeminal nerve 235. Therefore, the current flow may take place from one terminal of the lead 135 to an electrode such as electrode 226 (FIG. 2) through the tissue proximal to the trigeminal nerve 235, to a second electrode such as electrode 228 and a second terminal of the lead 135.

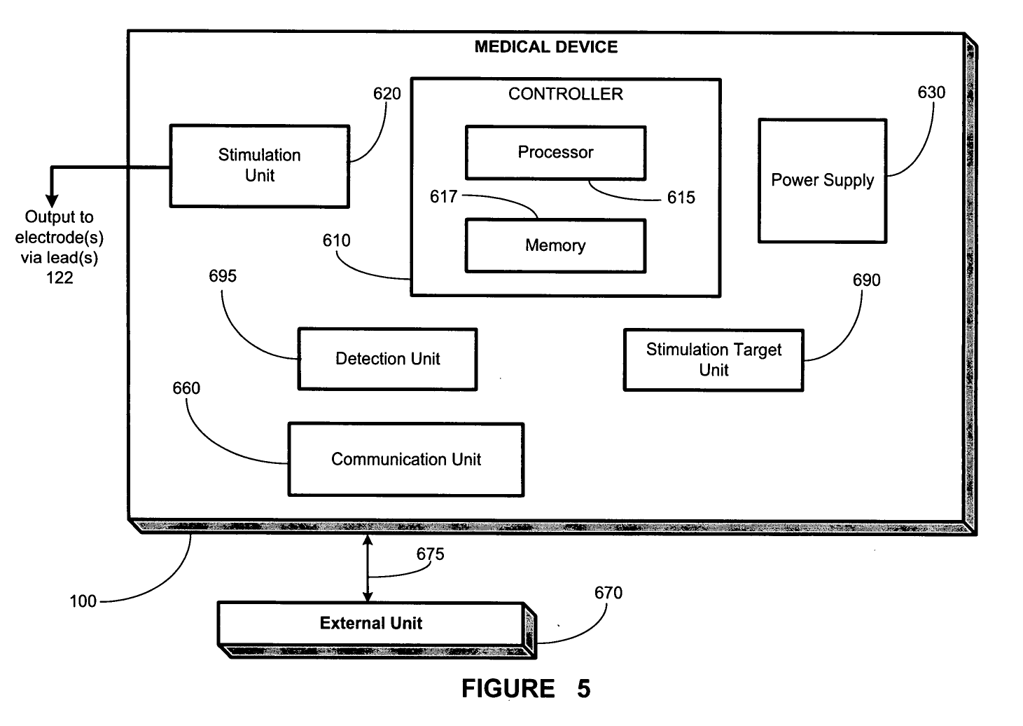

Turning now to FIG. 5, a block diagram depiction of the medical device 100, in accordance with an illustrative embodiment of the present invention is provided. The medical device 100 may comprise a controller 610 capable of controlling various aspects of the operation of the medical device 100. The controller 610 is capable of receiving internal data and/or external data and generating and delivering a stimulation signal to target tissues of the patient's body. For example, the controller 610 may receive manual instructions from an operator externally, or may perform stimulation based on internal calculations and programming. The controller 610 is capable of affecting substantially all functions of the medical device 100.

The controller 610 may comprise various components, such as a processor 615, a memory 617, etc. The processor 615 may comprise one or more microcontrollers, microprocessors, etc., that are capable of performing various executions of software components. The memory 617 may comprise various memory portions where a number of types of data (e.g., internal data, external data instructions, software codes, status data, diagnostic data, etc.) may be stored. The memory 617 may comprise random access memory (RAM) dynamic random access memory (DRAM), electrically erasable programmable read-only memory (EEPROM), flash memory, etc.

The medical device 100 may also comprise a stimulation unit 620. The stimulation unit 620 is capable of generating and delivering stimulation signals to one or more electrodes via leads. One or more leads 122 may be coupled to the medical device 100. Therapy may be delivered to the leads 122 by the stimulation unit 620 based upon instructions from the controller 610. The stimulation unit 620 may comprise various circuitry, such as stimulation signal generators, impedance control circuitry to control the impedance “seen” by the leads, and other circuitry that receives instructions relating to the type of stimulation to be performed. The stimulation unit 620 is capable of delivering a controlled current stimulation signal over the leads 122.

The medical device 100 may also comprise a power supply 630. The power supply 630 may comprise a battery, voltage regulators, capacitors, etc., to provide power for the operation of the medical device 100, including delivering the stimulation signal. The power supply 630 comprises a power-source battery that in some embodiments may be rechargeable. In other embodiments, a non-rechargeable battery may be used. The power supply 630 provides power for the operation of the medical device 100, including electronic operations and the stimulation function. The power supply 630 may comprise a lithium/thionyl chloride cell or a lithium/carbon monofluoride cell. Other battery types known in the art of medical devices may also be used.

The medical device 100 also comprises a communication unit 660 capable of facilitating communications between the medical device 100 and various devices. In particular, the communication unit 660 is capable of providing transmission and reception of electronic signals to and from an external unit 670. The external unit 670 may be a device that is capable of programming various modules and stimulation parameters of the medical device 100. In one embodiment, the external unit 670 is a computer system that is capable of executing a data-acquisition program. The external unit 670 may be controlled by a healthcare provider, such as a physician, at a base station in, for example, a doctor's office. The external unit 670 may be a computer, preferably a handheld computer or PDA, but may alternatively comprise any other device that is capable of electronic communications and programming. The external unit 670 may download various parameters and program software into the medical device 100 for programming the operation of the device. The external unit 670 may also receive and upload various status conditions and other data from the medical device 100. The communication unit 660 may be hardware, software, firmware, and/or any combination thereof. Communications between the external unit 670 and the communication unit 660 may occur via a wireless or other type of communication, illustrated generally by line 675 in FIG. 5.

The medical device 100 also comprises a detection unit 695 that is capable of detecting various conditions and characteristics of the gastrointestinal function(s) of a patient. For example, the detection unit 695 may comprise hardware, software, and/or firmware that are capable of determining data relating to at least one symptom of motion sickness. The detection unit 695 may comprise means for deciphering data from various sensors that are capable of measuring the factors described herein. Based upon the data deciphered by the detection unit 695, the medical device 100 may deliver stimulation to a portion of the autonomic nerve.

The medical device 100 may also comprise a stimulation target unit 690 that is capable of directing a stimulation signal to one or more electrodes that is operationally coupled to various portions of the autonomic nerves. The stimulation target unit 690 may direct a stimulation signal to a particular branch or location of the trigeminal nerve. Therefore, upon an onset of motion sickness, the medical device 100 may select various portions of the autonomic nerve described herein to stimulate to perform an afferent stimulation in order to alleviate motion sickness.

One or more blocks illustrated in the block diagram of medical device 100 in FIG. 5 may comprise hardware units, software units, firmware units and/or any combination thereof. Additionally, one or more blocks illustrated in FIG. 5 may be combined with other blocks, which may represent circuit hardware units, software algorithms, etc. Additionally, any number of the circuitry or software units associated with the various blocks illustrated in FIG. 5 may be combined into a programmable device, such as a field programmable gate array, an ASIC device, etc.

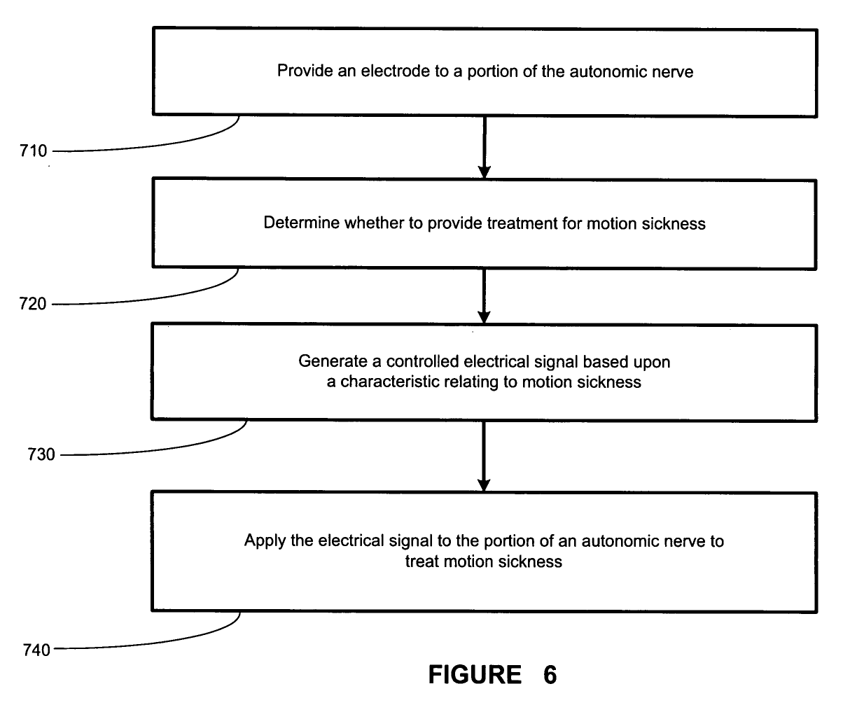

Turning now to FIG. 6, a flowchart depiction of a method for treating motion sickness, in accordance with one illustrative embodiment of the present invention is provided. An electrode may be transcutaneously coupled to a portion of a cranial nerve to perform a stimulation function and/or a blocking function to treat motion sickness. In one embodiment, one or more electrodes may be positioned to deliver a stimulation signal to a portion of the autonomic nerve (block 710). A determination may be made as to whether a treatment for motion sickness should be provided (block 720). In one embodiment, this determination may include receiving an external input (e.g., a magnetic input, a tap input, a wireless communications input, etc.) indicative of a request for treatment. In another embodiment, an automated sensing of an indication of motion sickness may be performed, prompting the determination to provide treatment. In yet another embodiment, an external input may trigger a detection algorithm to sense an indication of motion sickness, prompting a determination to provide treatment.

The medical device 100 may then generate a controlled electrical signal, based upon one or more characteristic relating to motion sickness(s) of the patient (block 730). This may include a predetermined electrical signal that is preprogrammed based upon a particular condition of a patient, such as data relating to at least one symptom of motion sickness. For example, a physician may pre-program the type of stimulation in order to treat the patient. The medical device 100 may then generate a signal, such as a controlled-current pulse signal.

The medical device 100 may then deliver the stimulation signal to the portion of the autonomic nerve (block 740). The application of the electrical signal may be delivered to any branch of the right and/or left trigeminal nerve, the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve. In one embodiment, application of the stimulation signal may be designed to promote an afferent effect to either attenuate or increase the activity of the vomiting/nausea response.

In another embodiment, application of the stimulation signal may be designed to promote a blocking effect relating to a signal that is being sent from the brain to the various portions of the gastrointestinal system to treat motion sickness. This may be accomplished by delivering a particular type of controlled electrical signal, such as a controlled current signal to the autonomic nerve. In yet another embodiment, afferent fibers may also be stimulated to treat motion sickness.

Additional functions, such as a detection process, may be alternatively employed with the embodiment of the present invention. The detection process may be employed such that an external detection and/or an internal detection of a bodily function may be used to adjust the operation of the medical device 100.

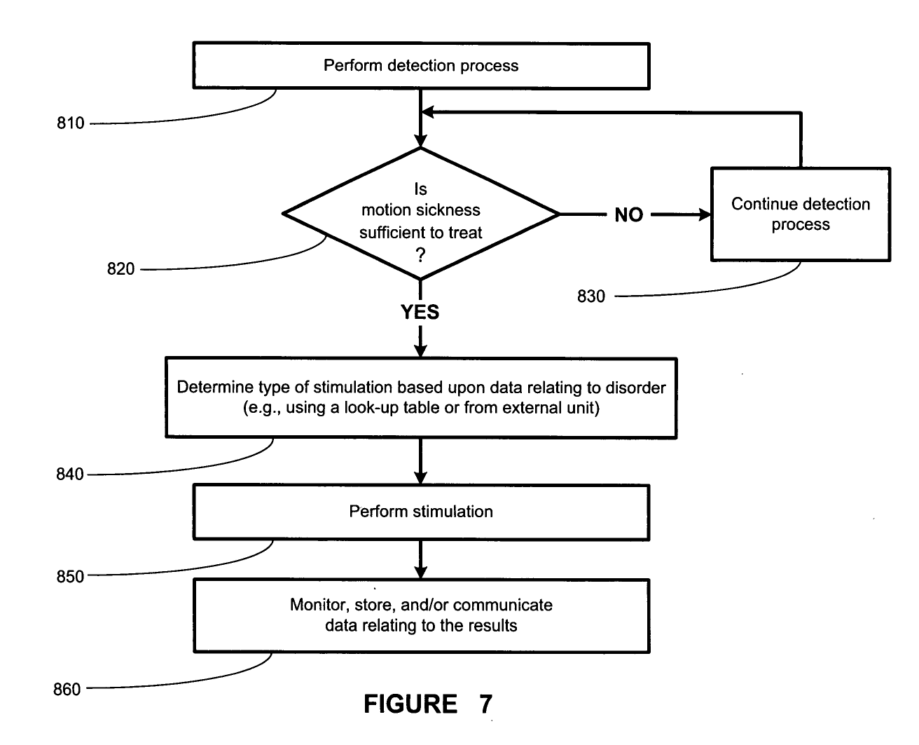

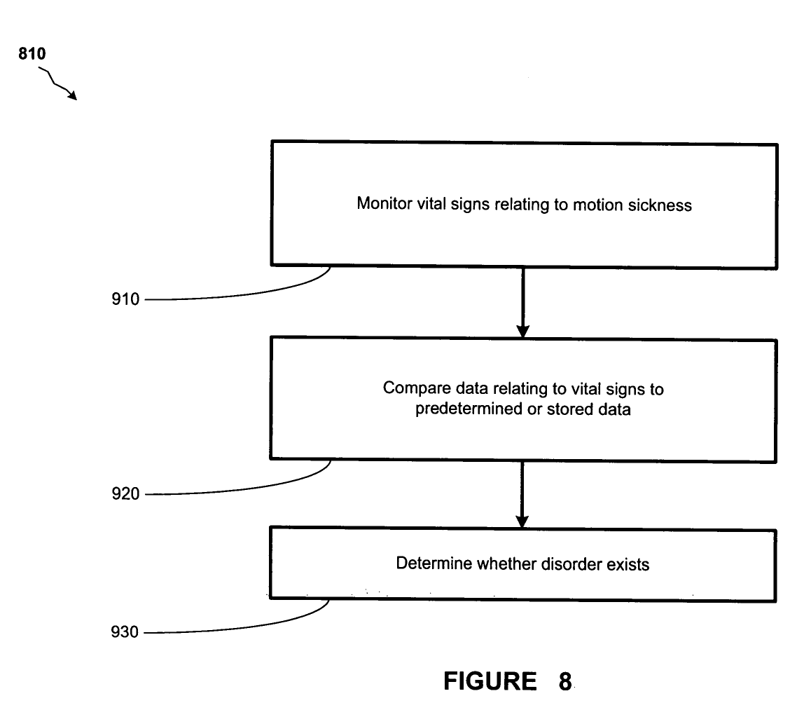

Turning now to FIG. 7, a block diagram depiction of a method in accordance with an alternative embodiment of the present invention is illustrated. The medical device 100 may perform a database detection process (block 810). The detection process may encompass detecting a variety of types of characteristics of motion sickness. A more detailed depiction of the steps for performing the detection process is provided in FIG. 8, and accompanying description below. Upon performing the detection process, the medical device 100 may determine whether a detected symptom of motion sickness is sufficiently severe to treat based upon the measurements performed during the detection process (block 820). This determination may be based on various factors, such as whether it is greater than a predetermined value where intervention by the medical device 100 is desirable. Upon a determination that the disorder is insufficient to treat by the medical device 100, the detection process is continued (block 830).

Upon a determination that the disorder is sufficient to treat using the medical device 100, a determination as to the type of stimulation based upon data relating to the disorder, is made (block 840). The type of stimulation may be determined in a variety of manners, such as performing a look-up in a look-up table that may be stored in the memory 617. Alternatively, the type of stimulation may be determined by an input from an external source, such as the external unit 670 or an input from the patient. Further, determination of the type of stimulation may also include determining the location as to where the stimulation is to be delivered. Accordingly, the selection of particular electrodes, which may be used to deliver the stimulation signal, is made. A more detailed description of the determination of the type of stimulation signal is provided in FIG. 10 and accompanying description below.

Upon determining the type of stimulation to be delivered, the medical device 100 may perform the stimulation by delivering the electrical signal to one or more selected electrodes (block 850). Upon delivery of the stimulation, the medical device 100 may monitor, store, and/or compute the results of the stimulation (block 860). For example, based upon the calculation, a determination may be made that adjustment(s) to the type of signal to be delivered for stimulation, may be performed. Further, the calculations may reflect the need to deliver additional stimulation. Additionally, data relating to the results of stimulation may be stored in memory 617 for later extraction and/or further analysis. Also, in one embodiment, real time or near real time communications may be provided to communicate the stimulation result and/or the stimulation log to an external unit 670.

Turning now to FIG. 8, a more detailed block diagram depiction of the step of performing the detection process of block 810 in FIG. 7, is illustrated. The system 100 may monitor one or more vital signs relating to the vestibular system and vomiting/nausea response of the patient (block 910). In another embodiment, these factors may be detected by external means, for example, by the patient or a healthcare provider making a subjective assessment of the need for treatment and providing same to the medical device 100 an external device via the communication system 660.

Upon acquisition of various vital signs, a comparison may be performed comparing the data relating to the vital signs to predetermined, stored data (block 920). Based upon the comparison of the collected data with theoretical, stored thresholds, the medical device 100 may determine whether a disorder exists (block 930). For example, various vital signs may be acquired in order to determine afferent fibers are to be stimulated. Based upon the determination described in FIG. 8, the medical device 100 may continue to determine whether the disorder is sufficiently significant to perform treatment, as described in FIG. 7.

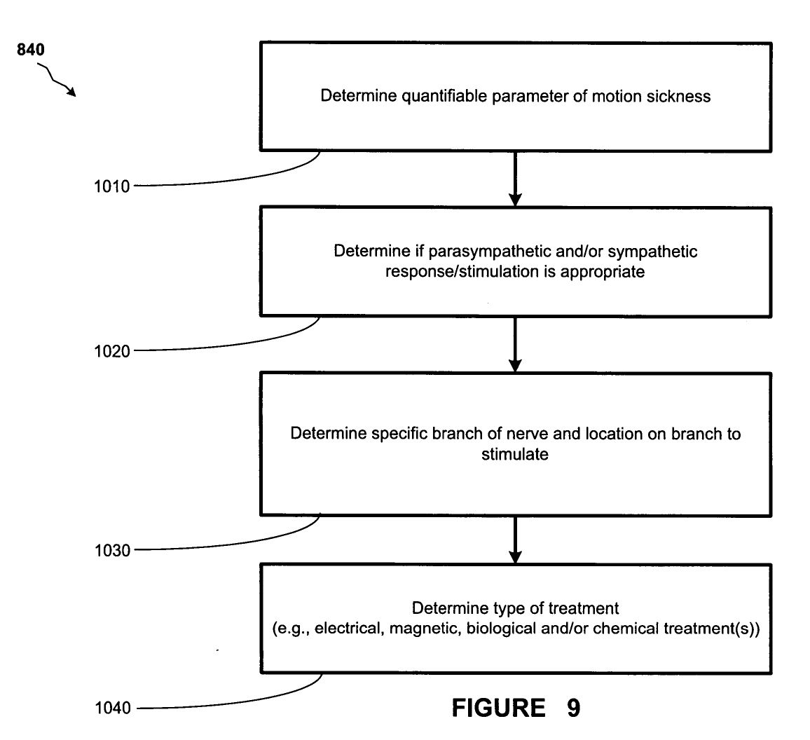

Turning now to FIG. 9, a more detailed flowchart depiction of the step of determining the type of stimulation indicated in block 840 of FIG. 7, is illustrated. The medical device 100 may determine a quantifiable parameter of motion sickness (block 1010). These quantifiable parameters, for example, may include a frequency of occurrence of various symptoms of motion sickness, such as yawning, hypersalivation, pallor, diaphoresis, or hyperventilation, among others.

Additionally, external devices may perform such calculation and communicate the results and/or accompanying instructions to the medical device 100, as shown in FIG. 9. The medical device 100 may also determine the specific batch of the nerve to stimulate (block 1030). For example, for a particular type of stimulation to be performed, the decision may be made to stimulate the main trunk of the right and/or left trigeminal nerve, the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve. The medical device 100 may also indicate the type of treatment to be delivered. For example, an electrical treatment alone or in combination with another type of treatment may be provided based upon the quantifiable parameter(s) that are detected (block 1040). For example, a determination may be made that an electrical signal by itself is to be delivered. Alternatively, based upon a particular type of disorder, a determination may be made that an electrical signal, in combination with a magnetic signal, such as transcranial magnetic stimulation (TMS) may be performed.

In addition to electrical and/or magnetic stimulation, a determination may be made whether to deliver a chemical, biological, and/or other type of treatment(s) in combination with the electrical stimulation provided by the medical device 100. In one example, electrical stimulation may be used to enhance the effectiveness of a chemical agent, such as nausea-reducing drug. Therefore, various drugs or other compounds may be delivered in combination with an electrical stimulation or a magnetic stimulation. Based upon the type of stimulation to be performed, the medical device 100 delivers the stimulation to treat motion sickness.

All of the methods and apparatus disclosed and claimed herein may be made and executed without undue experimentation in light of the present disclosure. While the methods and apparatus of this invention have been described in terms of particular embodiments, it will be apparent to those of skill in the art that variations may be applied to the methods and apparatus and in the steps or in the sequence of steps of the method described herein without departing from the concept, spirit and scope of the invention as defined by the appended claims. It should be especially apparent that the principles of the invention may be applied to selected cranial nerves other than the trigeminal nerve to achieve particular results.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. A method of treating motion sickness in a patient, comprising:
coupling at least one electrode to the patient at least one location in proximity to at least one cranial nerve; and
applying an electrical signal to said cranial nerve using said electrode to treat said motion sickness.

2. The method of claim 1, wherein the cranial nerve is selected from the group consisting of the trigeminal nerve and a trigeminal nerve branch.

3. The method of claim 2, wherein the trigeminal nerve branch is selected from the group consisting of the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve.

4. The method of claim 1, wherein treating said motion sickness comprises treating motion sickness induced by at least one of automobile travel, boat travel, aircraft travel, and spacecraft travel.

5. The method of claim 1, further comprising the steps of:
providing a programmable electrical signal generator;
coupling said signal generator said at least one electrode;
generating an electrical signal with the electrical signal generator; and
applying the electrical signal to the electrode.

6. The method of claim 5, further comprising programming the electrical signal generator to define the electrical signal by at least one parameter selected from the group consisting of a current magnitude, a pulse frequency, a pulse width, a pulse period, an on-time and an off-time, wherein said at least one parameter is selected to treat the motion sickness.

7. The method of claim 6, wherein said at least one parameter is characterized by a value that varies randomly within a defined range from pulse to pulse.

8. The method of claim 1, further comprising detecting a symptom of the motion sickness, and wherein applying the electrical signal is initiated in response to detecting said symptom.

9. The method of claim 8, wherein detecting the symptom is performed by the patient.

10. The method of claim 1, wherein applying the electrical signal comprises applying said signal to the nerve branch using said at least one electrode during a first treatment period, and said method further comprises applying a second electrical signal to the nerve branch using said at least one electrode during a second treatment period, to treat the motion sickness.

11. A method of treating motion sickness in a patient, comprising:
coupling a first electrode to the skin of the patient at a first location in proximity to at least a first cranial nerve; and
coupling a second electrode to the skin of the patient at a second location in proximity to at least a second cranial nerve; and
applying a first electrical signal to said first cranial nerve using said first electrode during a first time period and applying a second electrical signal to said second cranial nerve using said second electrode during a second time period to treat said motion sickness.

12. The method of claim 11, wherein the first and second cranial nerve are selected from the group consisting of the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve.

13. The method of claim 11, wherein said at least a first nerve branch and said at least a second nerve branch comprise the same nerve branch.

14. A method of treating motion sickness in a patient, comprising:
receiving a signal indicative of a symptom of the motion sickness in said patient; and
providing an electrical signal to an electrode coupled to the patient at least one location in proximity to at least one cranial nerve to treat said motion sickness in response to said signal.

15. The method of claim 14, wherein the cranial nerve is selected from the group consisting of the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve

16. The method of claim 14, wherein receiving a signal indicative of a symptom of the motion sickness in said patient comprises using a sensor to receive said signal indicative of a symptom of the motion sickness in said patient.

17. The method of claim 14, wherein receiving a signal indicative of a symptom of the motion sickness in said patient comprises receiving said signal from a source external to the patient's body.

18. A medical device for treating motion sickness in a patient, comprising:
an interface for receiving a signal indicative of a symptom of the motion sickness in said patient;
an electrode coupled to the patient at least one location in proximity to at least one cranial nerve, and
a controller for providing an electrical signal to said electrode to treat said motion sickness based upon said signal.

19. The medical device of claim 18, wherein the electrode is coupled to a cranial nerve branch selected from the group consisting of the infraorbital branch of the trigeminal nerve, the buccal branch of the trigeminal nerve, the mental branch of the trigeminal nerve, the supratrochlear branch of the trigeminal nerve, the supra-orbital branch of the trigeminal nerve, the infratrochlear branch of the trigeminal nerve, the external nasal branch of the trigeminal nerve, the auriculotemporal branch of the trigeminal nerve, the zygomaticofacial branch of the trigeminal nerve, and the palpebral branch of the trigeminal nerve

20. The medical device of claim 18, further comprising a sensor to sense a symptom of the motion sickness in said patient and provide said signal indicative of a symptom of the motion sickness in said patient.

21. The medical device of claim 18, wherein said signal indicative of a symptom of the motion sickness in said patient is provided by a source external to the patient's body.

22. The method of claim 1, further comprising:
coupling a drug delivery device in direct or indirect fluid communication with the patient's bloodstream; and
applying a drug to said bloodstream using said drug delivery device to treat said motion sickness.

23. The method of claim 22, wherein the drug is selected from the group consisting of promethazine, dimenhydrinate, diphenhydramine, meclizine, cyclizine, diazepam, and scopolamine.
